(19) United States
(12) Patent Application Publication
(10) Pub. No.: US 2004/0167408 A1
(43) Pub. Date: Aug. 26, 2004

(54) Medicine case, medicine case with blood pressure measuring function, medication management system, and blood pressure monitor

(75) Inventors: Tameo Ashida, Osaka (JP); Norihito Yamamoto, Otsu-shi (JP); Yoshihiko Sano, Kyoto-shi (JP); Kazuhisa Tanabe, Kyoto-shi (JP); Yutaka Imai, Sendai-shi (JP)

Correspondence Address: MORRISON & FOERSTER LLP, 1650 TYSONS BOULEVARD, SUITE 300, MCLEAN, VA, 22102, US

(73) Assignees: OMRON HEALTHCARE CO., LTD., Kyoto-shi (JP); Yutaka IMAI, Sendai-shi (JP)

(21) Appl. No.: 10/729,375

(22) Filed: Dec. 8, 2003

(30) Foreign Application Priority Data
Dec. 6, 2002 (JP) 2002-354927

(51) Int. Cl. A61B 5/02

(52) U.S. Cl. 600/485; 206/538; 221/2

(57) ABSTRACT

Provided are a medicine case, a medicine case with a blood pressure measuring function, and a medication management system capable of storing blood pressure measurement together with medication record automatically, displaying simultaneously, and entering and issuing, and also supplying clinically useful information. In the medicine case, since the medicine to be taken is indicated at every timing specified by the attending physician, and the medicine can be taken without error or fail, and also the medication information and blood pressure measuring results are stored and displayed simultaneously, and can be observed simultaneously at a glance at a medical institute, and changes in blood pressure information and medication effects are determined easily, so that clinically useful information can be obtained.

### BACKGROUND OF THE INVENTION

1. Field of the Invention

The present invention relates to a medicine case, a medicine case with a blood pressure measuring function, and a medication management system and, more particularly, to a medicine case capable of detecting medication of the user, storing the medication, displaying the medication record, and issuing to outside, a medicine case with a blood pressure measuring function capable of displaying the medication record together with the blood pressure value measured by the user and issuing to outside, and a medication management system and a blood pressure monitor using the medicine case with a blood pressure measuring function.

2. Description of the Related Art

When classified by disease of patients, hypertension tops the list in age group of 40 years and over, and more than majority of people over 65 years of age are said to be hypertensive. To treat this ubiquitous disease of hypertension, a rapid progress is made in the investigation of pathology and therapy, and excellent hypotensive medicines are making dramatic effects.

However, the major problem of this medicine therapy is failure of treatment due to patient's negligence in medication. Not only the patient does not take the medicine correctly, but also the attending physician fails to know this fact, and unsuccessful cases of hypotensive medication are commonplace.

For hypertensive patients, in particular, outpatients, conformity to medication is difficult. That is, it is troublesome to take the prescribed medicine every day at specified time, and it is almost impossible to record the medication together with the blood pressure measured by oneself. In addition, many patients do not report correctly to the attending physicians.

In the light of such background, there is an increasing demand for development of tool and system capable of forcing the patient to take the medicine at specified time at home, storing the medication record automatically, or storing the medication record and blood pressure value, and reporting such information to the attending physician.

In order to solve such problems, various methods have been proposed so far.

For example, a medicine case (see Japanese Unexamined Patent Publication No. 7-236681 (1995)) comprises a plurality of compartments storing medicines, having lids indicating the date and time of medication. As a result, failure of taking medicine or duplicate medication can be prevented.

In other medication management apparatus and method (see Japanese Unexamined Patent Publication No. 2001-206463), the time is printed on the printing paper every time the medicine is taken out of the case.

In a medical data processing system (see Japanese Examined Patent Publication No. 7-52459 (1995)), the blood pressure value and medication information of patients treated by dialysis are stored and printed.

In an automatic blood pressure measuring apparatus (see Japanese Utility Model Registration No. 3004300), together with the blood pressure values, the information of the prescribed medicine, that is, the prescription of the medicine, purpose of administration and cautions are displayed or issued. Therefore, the physician is not required to describe the prescribed medication.

In the medicine case of Japanese Unexamined Patent Publication No. 7-236681 (1995), the medication record is not stored automatically or displayed, and execution of medication cannot be confirmed.

In the medication management apparatus and method of Japanese Unexamined Patent Publication No. 2001-206463, the information including the type of the medicine taken cannot be stored, displayed or issued to outside, it is ineffective for prevention of in compliance of medication.

In the medical data processing system of Japanese Examined Patent Publication No. 7-52459 (1995), the blood pressure value and medication information are entered by reading by the image scanner by the OCR apparatus, not entered automatically, and the operation is complicated, it is designed as a system for patients treated by dialysis in hospital, not for patients measuring at home.

In the automatic blood pressure measuring apparatus of Japanese Utility Model Registration No. 3004300, since the medication result is not recorded or issued, the information obtained by this apparatus was meaningless clinically.

### SUMMARY OF THE INVENTION

The invention is devised in the light of the above problems, and it is hence an object of the invention to provide a medicine case, a medicine case with a blood pressure measuring function, a medication management system, and a blood pressure monitor capable of recording and displaying the medication record automatically, entering the medication instruction from outside and issuing the medication record to outside, displaying the measured blood pressure value together with medication record simultaneously, and issuing outside, and providing clinically useful information.

In order to achieve the object, the medicine case of the invention comprises: medicine storage means for storing one or plural medicines to be taken each time by the user in divided portions; medication instruction storage means for storing instruction of medication entered from outside; medication instruction means for instructing the medicine to be taken from the divided portions stored in the medicine storage means on the basis of the medication instruction of the medication instruction storage means; medication detection means for detecting taking of the medicine instructed by the medication instruction means; storage output means for storing the medicine taking record detected by the medication detection means, and issuing to outside; and display means for displaying the medicine taking record stored in the storage output means.

The medicine storage means is constituted of a plurality of compartments having lids for storing in divided portions according to each medicine taking timing by the user, the medication instruction means instructs the compartment storing the medicine to be taken, and the medication detection means can detect opening and closing of the lid of the compartment.

The medicine storage means is constituted of a plurality of slits holding one pouch each of the medicine to be taken, the medication instruction means instructs one or plural slits holding the pouches to be taken, and the medication detection means detects taking of the pouch out of the slit.

Therefore, the medicine to be taken at the timing specified by a medical institute or the like is stored and indicated at every timing, and the medicine can be taken without error, and failure of treatment by in compliance of medication can be improved, and the medication record can be detected, recorded and displayed automatically, so that an accurate medication record is obtained.

The medicine case with a blood pressure measuring function of the invention further comprises, in addition to the medicine case, blood pressure measurement means for measuring the blood pressure of the user, and the storage output means includes storage output means for storing the blood pressure value measured by the blood pressure measurement means and the medicine taking record detected by the medication detection means, and issuing to outside, and display means for displaying the blood pressure value and the medicine taking record stored in the storage output means.

The storage output means of the medicine case with a blood pressure measuring function stores the systolic blood pressure value, diastolic blood pressure value, pulse rate and date and time of measurement measured by the blood pressure measurement means, and the medicine taking time detected by the medication detection means, and also issues to outside.

The display means of the medicine case with a blood pressure measuring function displays the systolic blood pressure value, diastolic blood pressure value, pulse rate, date and time of measurement, type of medicine, and medicine taking time stored in the storage output means, in list display, graph display, or printer display.

Therefore, the blood pressure information and medication record indispensable for medicine therapy of hypertension can be obtained simultaneously at a glance, and it is easy to understand the changes of blood pressure information and medication effects, which encourages the user to comply with the medication.

The medication management system with a blood pressure measuring function of the invention comprises, in addition to the medicine case with a blood pressure measuring function, medication instruction input means for entering medication instruction into the medication instruction storage means, and external display means for storing and displaying the storage information issued from the storage output means.

Further, the medication instruction input means and external display means are terminal controllers installed at a medical institute, and it may further comprise communication means for transmitting and receiving between the terminal controllers and the medicine case with a blood pressure measuring function via public lines.

Therefore, the physician at a medical institute simultaneously acquires the blood pressure information and medication record, and obtains useful information for clinical purpose, and can make further medication instructions, so that an effective hypotensive treatment by medicine can be executed.

The blood pressure monitor of the invention comprises, in addition to the conventional blood pressure measurement function, medication information storage means for receiving and storing medication information such as the presence/absence of medication, the kind of medicines to be taken, and date and time of medication from outside, and display means for displaying the medication information stored in the medication information storage means together with the blood pressure values and date and time of measurement.

Therefore, while measuring the blood pressure by the blood pressure monitor, not only the blood pressure values but also the medication record can be obtained simultaneously at a glance, and it is easier for the user of the blood pressure monitor to understand the medication effects, and the daily measurement of blood pressure is encouraged and promoted.

### BRIEF DESCRIPTION OF THE DRAWINGS

FIG. 1 is an outline view of a medication management case in an embodiment of a medicine case of the invention;

FIG. 2a is a sectional view of taken along line A-A in FIG. 1; in which shows a state of opening a lid

FIG. 2b is a sectional view of taken along line A-A in FIG. 1; in which shows a state of closing a lid

FIG. 3 is an outline view of a medication management case in another embodiment of the medicine case of the invention;

FIG. 4 is an outline view of a medication management case with a blood pressure monitor in an embodiment of a medicine case with a blood pressure measuring function of the invention;

FIG. 5 is an outline view of a medication management case with a blood pressure monitor in another embodiment of the medicine case with a blood pressure measuring function of the invention;

FIG. 6 is a functional block diagram showing a configuration of a medication management case with a blood pressure monitor in an embodiment of a medicine case with a blood pressure measuring function of the invention;

FIG. 7 is a schematic diagram showing an actual status of use of the medication management case with a blood pressure monitor;

FIG. 8 is a flowchart showing procedure of data processing at a medical institute in the status of use of the medication management case with a blood pressure monitor;

FIG. 9 is a flowchart showing procedure of data processing at home in the status of use of the medication management case with a blood pressure monitor;

FIG. 10 is an example of display of medication record and blood pressure measurement value in an display of the medication management case with a blood pressure monitor;

FIG. 11 is an example of graph display of medication record and blood pressure measurement in an display of the medication management case with a blood pressure monitor;

FIG. 12 is a diagram showing an example of configuration of the medication management system of the invention; and

FIG. 13 is a functional block diagram showing a configuration of a blood pressure monitor with a medication information display function in an embodiment of a blood pressure monitor of the invention.

### DESCRIPTION OF THE PREFERRED EMBODIMENTS

Referring now to the drawings, a preferred embodiment of the invention will be described below.

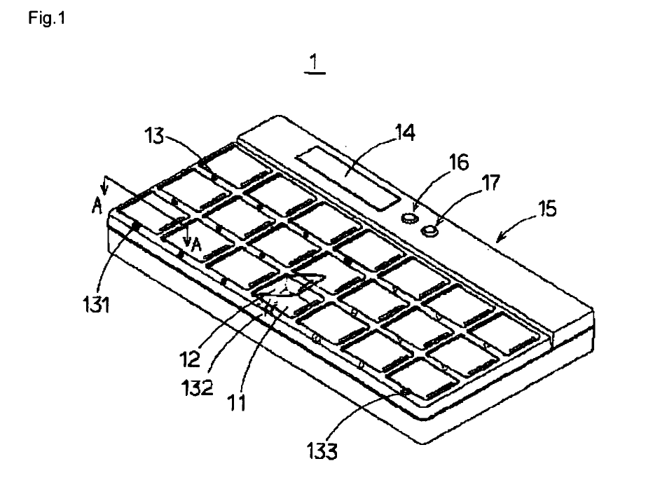

FIG. 1 is an outline view of a medication management case 1 as an embodiment of a medicine case of the invention.

This medication management case 1 comprises a plurality of compartments 11 having lids 12 as medicine storage means, and one or plural medicines to be taken each time by the user are put in each compartment 11. Near the compartment 11, there is a medication instruction LED 13 for indicating the compartment containing the medicine to be taken, and this embodiment has three kinds of display forms depending on the medication timing. That is, a medication instruction LED 131 for the compartment already consumed is lit dimly, a medication instruction LED 132 for the compartment of the exact medication timing is flickering, and a medication instruction LED 133 for the compartment not ready for medication is not lit.

The medication management case 1 has a display 14, which indicates the present time and the medication status. At the back side 15 of the medication management case 1, although not shown, a power switch and an external communication I/F are provided. There are also an external communication LED 16 showing external communication through this external communication I/F and an external output button 17 to be pressed at the time of output of medication information to outside.

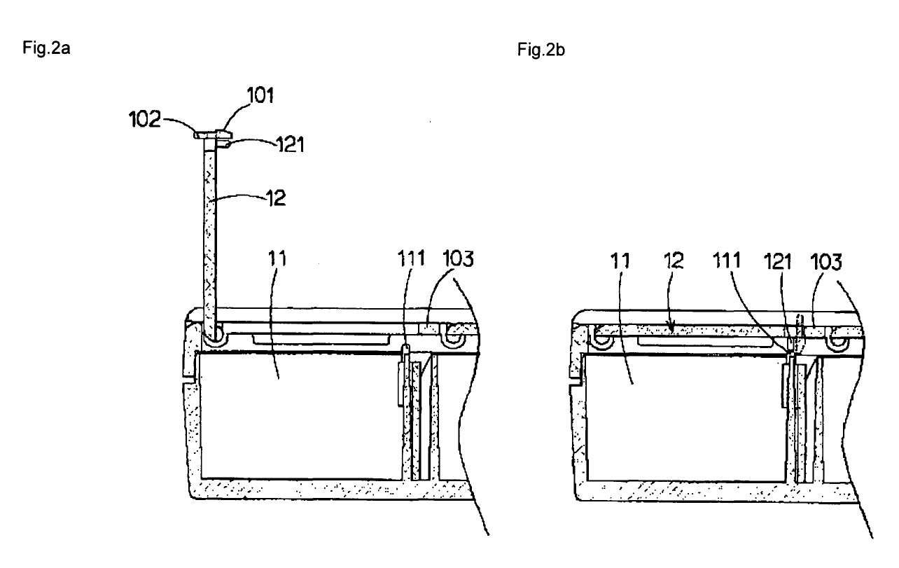

FIG. 2a and FIG. 2b are a sectional view taken along line A-A in FIG. 1 for describing the medication detection means, in which FIG. 2a shows a state of opening a lid 12, and FIG. 2b shows a state of closing the lid 12. As the medication detection means, the lid 12 has a protrusion 121, and the compartment 11 has a small switch 111 at a position corresponding to the protrusion 121 of the lid 12, and opening or closing of the lid is detected by the protrusion 121 and small switch 111. At the end portion of the lid 12, a pawl 101 and a protruding portion 102 are disposed, and while the lid 12 is closed: (b), the pawl 101 is matched so as to be hooked on a pawl insertion portion 103. When opening the lid 12, by moving the protruding portion 102 integrated with the pawl 101, the pawl 101 is separated from the pawl inserting portion 103, and the lid 12 can be opened, and it is designed so that the lid 12 may not be opened easily.

More preferably, a further mechanism may be provided so that the lid of the compartment at the medication timing can be opened, but the lid of the already consumed compartment and the lid of the compartment not ready for medication cannot be opened or closed.

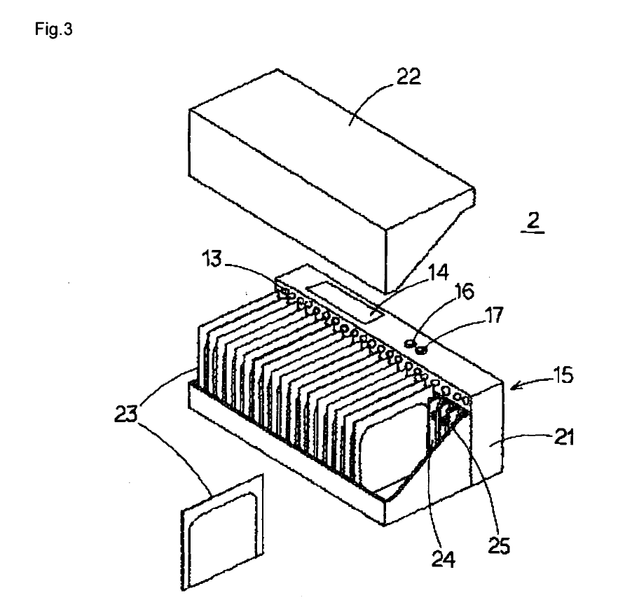

FIG. 3 is an outline view of a medication management case 2 as another embodiment of the medicine case of the invention. This medication management case 2 is constituted of a main body 21, and a cover 22. As shown in FIG. 3, when the cover 22 is removed from the main body 21 of the medication management case 2, slits 24 are disposed as medicine storage means, and a pouch 23 containing the medicine to be taken is held in each one of the plural slits 24. As medication detection means, a photo sensor 25 for detecting presence or absence of pouch is provided in each slit 24.

As medication instruction means, a medication instruction LED 13 is disposed near each slit. By lighting of the medication instruction LED 13, it is instructed same as in the medication management case in the embodiment, but if plural pouches are to be taken, the medication instruction LEDs 13 of plural corresponding pouches are lit.

The main body 21 of the medication management case 2 has a display 14 for displaying the present time and the medication status, and a power switch and an external communication I/F are provided at the back side 15 of the medication management case 2. There are also an external communication LED 16 and an external output button 17, same as in the case of the medication management case 1.

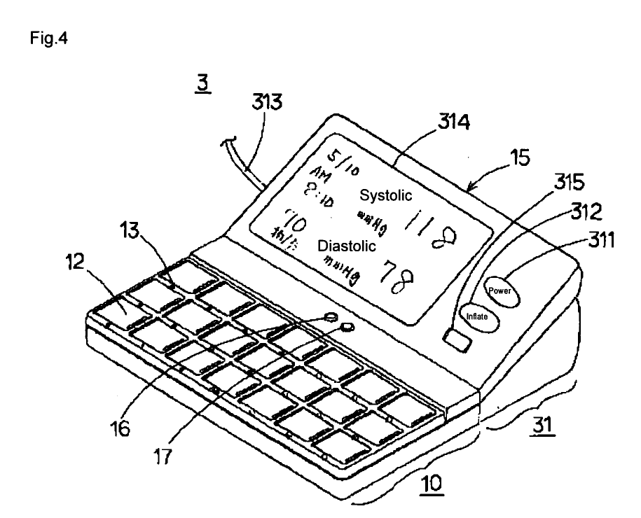

FIG. 4 is an outline view of a medication management case 3 with a blood pressure monitor as an embodiment of a medicine case with a blood pressure measuring function of the invention.

The medication management case 3 with a blood pressure monitor comprises a medication management case 10 and a blood pressure monitor 31. The blood pressure monitor 31 includes a power switch 311, an inflation switch 312, and a tube 313 to be connected to a cuff (not shown). The blood pressure measuring function of the blood pressure monitor 31 is same as in the conventional brachial blood pressure monitor, and its description will not be given. A display 314 of the medication management case 3 with a blood pressure monitor displays not only the measured blood pressure values, but also the medication record, together with the blood pressure value, on the same screen by means of a display changeover switch 315. The display contents will be described later.

At the back side 15 of the medication management case 3 with a blood pressure monitor, there is a connector (not shown) to be connected to an external personal computer.

The medication management case 10 is almost same as the medication management case 1 mentioned above. However, the medication status is displayed in the display 314 of the medication management case 3 with a blood pressure monitor, and the medication management case 10 does not include the display 14.

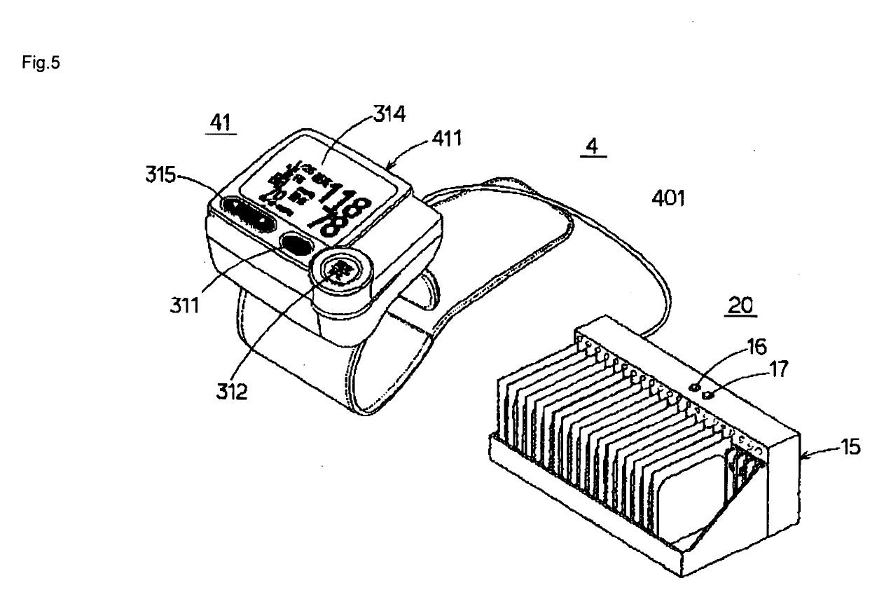

FIG. 5 is an outline view of a medication management case 4 with a blood pressure monitor as another embodiment of the medicine case with a blood pressure measuring function of the invention.

The medication management case 4 with a blood pressure monitor comprises a medication management case 20 and a blood pressure monitor 41. The medication management case 20 and the blood pressure monitor 41 are coupled by means of a communication cable 401. Coupling of the medication management case 20 and the blood pressure monitor 41 is not limited to the communication cable 401 or wire, but may include wireless means such as infrared ray. At the back side 411 of the blood pressure monitor 41, there is a connector (not shown) for connecting the communication capable 401. The blood pressure monitor 41 includes a power switch 311, an inflation switch 312, and the blood pressure measuring function of the blood pressure monitor 41 is same as in the conventional wrist blood pressure monitor, and its description will not be given. A display 314 of the blood pressure monitor 41 displays not only the measured blood pressure values, but also the medication record, together with the blood pressure value, on the same screen by means of a display changeover switch 315.

The medication management case 20 is almost same as the medication management case 2 mentioned above; therefore, its description will not be given. However, the medication status is displayed in the display 314 of the medication management case 3 with a blood pressure monitor, and it does not include the display 14. Further, at the back side 18 of the medication management case 2, although not shown in the figure, a connector (not shown) is provided for coupling with the communication cable 401.

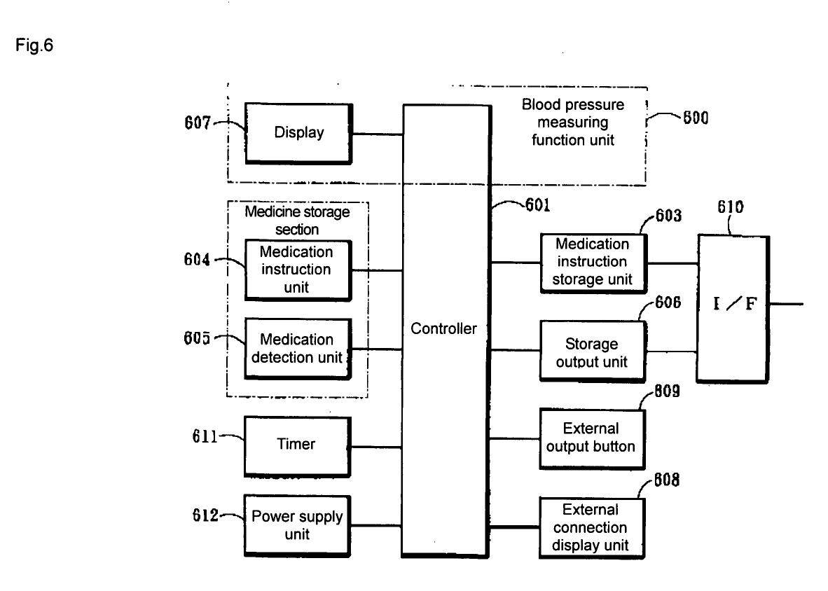

FIG. 6 is a functional block diagram showing the configuration of medication management case 3 with a blood pressure monitor as an embodiment of a medicine case with a blood pressure measuring function of the invention.

It is described below while referring to FIG. 6. A blood pressure measuring function section 600 of the medication management case 3 with a blood pressure monitor is same as the conventional brachial blood pressure monitor; therefore, its description will not be given. The medication management case 3 with a blood pressure monitor comprises a controller 601 constituted of CPU and others for controlling the entire medication management case, a medicine storage section 602 constituted of compartments 12 for holding the medicines to be taken, a medication instruction storage unit 603 storing the medication instruction entered from outside, a medication instruction unit 604 provided in the medicine storage section 602 for indicating the medicine to be taken according to the medication instruction of the medication instruction storage unit 603, a medication detection unit 605 for detecting taking of the medicine indicated by the medication instruction means 604, a storage output unit 606 for storing the medicine taking record detected by the medication detection unit 605 and various blood pressure measuring results, and issuing to outside, a display unit 607 for displaying the medicine taking record and the present time together with various blood pressure measuring results, an external connection display unit 608 constituted of a blood pressure monitor coupled LED 15 showing presence or absence of coupling with the blood pressure monitor and an external communication LED 16 for indicating communication with outside, an output button 609 for issuing the medicine taking record stored in the storage output unit 606 to outside, a timer 611, a power supply 612 as the entire power source of the medication management case 3 with a blood pressure monitor, and an I/F (interface) unit 613 for communication with outside.

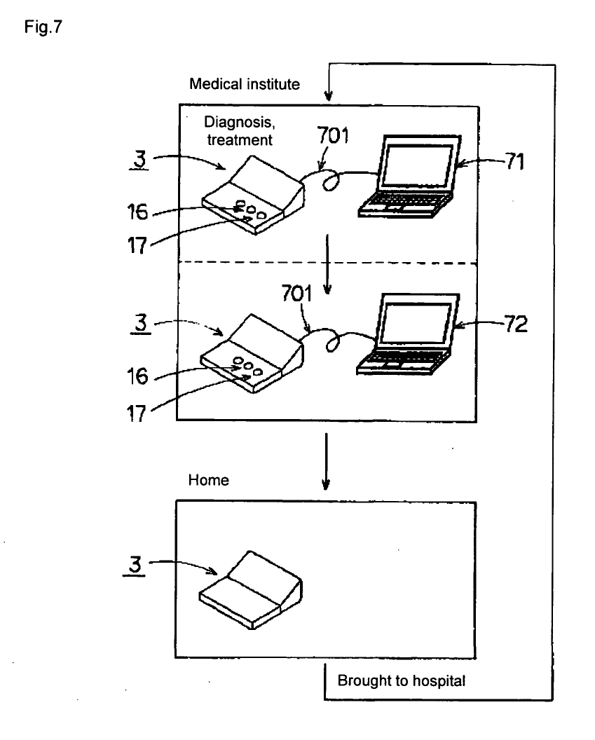

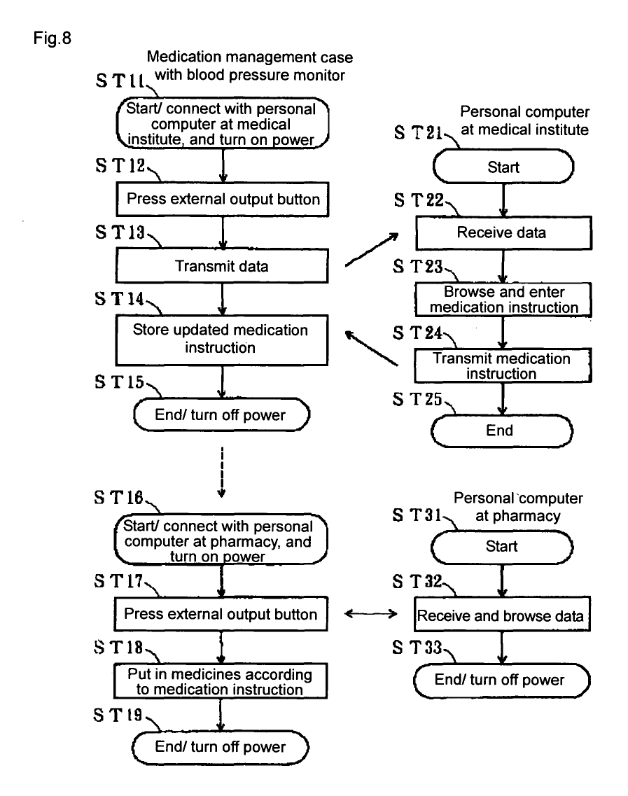

FIG. 7 is a schematic diagram showing an actual status of use of the medication management case 3 with a blood pressure monitor, and FIG. 8 is a flowchart showing the procedure of data processing at a medical institute using the medication management case 3 with a blood pressure monitor. The detail is described by referring to FIGS. 7 and 8.

The user brings the medication management case 3 with a blood pressure monitor to a hospital or medical institute of the attending physician. At the medical institute, the attending physician receives the medication management case 3 with a blood pressure monitor from the user, and plugs to a personal computer 71 at the medical institute by means of a coupling cable 701 (ST 11 in FIG. 8), and makes sure the external connection LED 16 of the medication management case 3 with a blood pressure monitor is lit, and presses the external output button 17 (ST 12).

As a result, the medication record and various blood pressure measuring results stored in the storage output unit 606 of the medication management case 3 with a blood pressure monitor can be browsed on the personal computer 71 of the medical institute. This system is a general method of exchanging information with the connected device, and the communication can be also made through a server although not shown. The attending physician observes the medication record and various blood pressure measuring results of the user, and enters the type of the medicine to be taken and medication timing in the personal computer 71 of the medical institute (ST 23). The input information is transmitted to the medication management case 3 with a blood pressure monitor (ST 24), and stored in the medication instruction storage unit 603 (ST 14). Then, unplugging from the personal computer 71 of the medical institute (ST 15), the medication management case 3 with a blood pressure monitor is returned to the user.

The user brings the medication management case 3 with a blood pressure monitor to the pharmacy of the medical institute or other specified pharmacy. At the pharmacy, same as in the case of the medical institute, the medication management case 3 with a blood pressure monitor and a personal computer 72 at the pharmacy are coupled by a communication cable 701 (ST 16), and the type of the medicine andmedication timing stored in the medication instruction storage unit 603 are browsed on the personal computer 72 of the pharmacy (ST 17). At the pharmacy, according to the observed type of medicine and medication timing, the prescribed medicines are put into the medicine storage section 602 of the medication management case 3 with a blood pressure monitor (ST 18). After putting in the medicines, unplugging from the personal computer 72 of the pharmacy, the medication management case 3 with a blood pressure monitor is returned to the user.

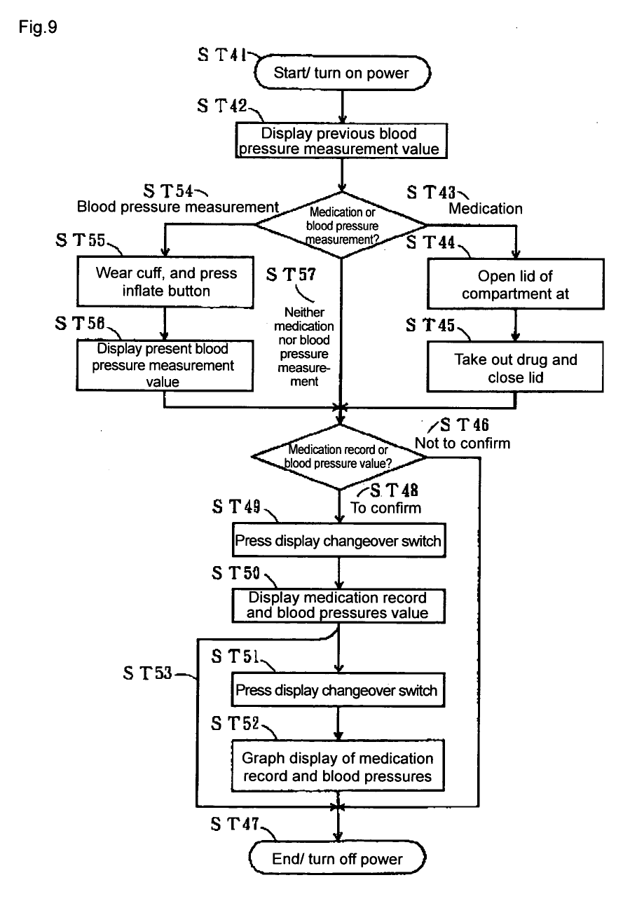

The user brings back the medication management case 3 with a blood pressure monitor to home, and takes the medicine or measures the blood pressure. FIG. 9 is a flowchart showing procedure of use at home of the medication management case 3 with a blood pressure monitor. It will be described by referring to FIG. 9.

First, a power switch 311 of the medication management case 3 with a blood pressure monitor is pressed (ST 41), and the display 314 shows the previous blood pressure measurement values (ST 42). When taking the medicine (ST 43), the medication instruction LED 132 of the compartment 11 at the medication timing is flickering, and the lid 12 of the compartment 11 is opened (ST 44), and the medicine is taken out, and the lid 12 is closed (ST 45). This opening and closing process of the lid 12 is detected by the medication detection unit 605, and is stored in the storage output unit 606 as medication record.

When no change is found in the hitherto medication record and blood pressure measurements (ST 46), the power switch 311 is pressed (ST 47) and the power is turned off.

If any change is found in the hitherto medication record or blood pressure value measurements (ST 48), the display changeover switch 315 is pressed (ST 49), and the display 314 shows the hitherto medication record and blood pressure measurement values (ST 50), and when the display changeover switch 315 is pressed again (ST 51), the display 314 shows a graph of blood pressure measurement values together with the medication record (ST 52). The contents of display will be described later. After confirming the display (ST 50), the power switch 311 is finally pressed (ST 47) and the power source is cut off. After the display of medication record and blood pressure measurements (ST 50), if graph display (ST 52) is not necessary (ST 53), after confirming the display (ST 50), the power switch 311 is pressed (ST 47) and the power source is turned off.

When measuring the blood pressure (ST54), the cuff (not shown) is worn and the inflation switch 312 is pressed (ST 55). This blood pressure measuring step is same as in the conventional brachial blood pressure monitor; therefore, its description will not be given. When the measurement is over, same as in the conventional blood pressure monitor, the measured blood pressure values are shown on the display 314 (ST 56).

If no change is found in the medication record or blood pressure measurement value up to the next step (ST 46), or when confirming (ST 48), the procedure is same as mentioned above. Further, without taking medicine or measuring the blood pressure (ST 57), the hitherto medication record and blood pressure measurement can be confirmed only (ST 48). The steps after ST 48 are same as mentioned above.

The contents of the medication record and blood pressure measurement value shown in the display 314 will be described below.

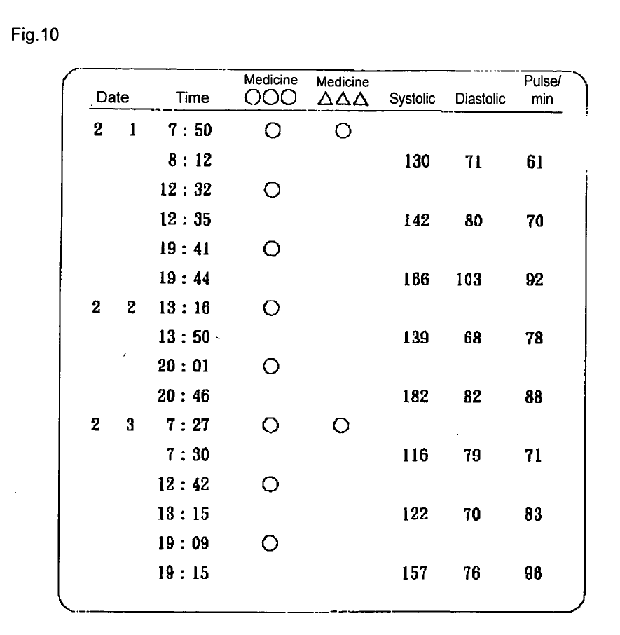

FIG. 10 is an example of display of medication record and blood pressure measurement value shown in the display 314 of the medication management case 3 with a blood pressure monitor.

The date and time in FIG. 10 shows the time of opening the lid 12 of the pertinent compartment 11 stored in the storage output unit 606 of the medication management case 3 with a blood pressure monitor, and the medicine name is the name of the contained medicine. This example instructs to take medicines three times a day, and specified to take two medicines in the morning among three times. The blood pressure value shows the systolic blood pressure value, diastolic blood pressure value, and pulse rate together with the date and time of measurement. This example of display in FIG. 10 shows that medication and blood pressure measurement were skipped in the morning of February 2.

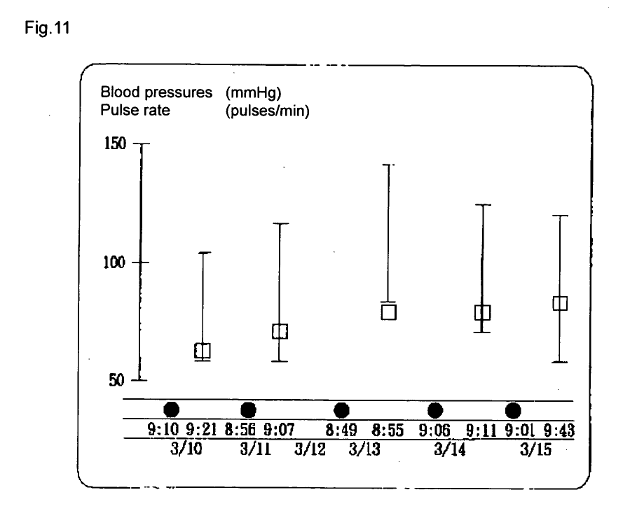

FIG. 11 is an example of graph display of medication record and blood pressure measurement value shown in the display 14 of the medication management case 3 with a blood pressure monitor. This graph display example does not show the example in FIG. 10, but shows an example of medication of one medicine once a day after breakfast and measurement of blood pressure once a day after breakfast. The lower column of the diagram shows the time of medication and time of blood pressure measurement together with the date, and the bullet shows taking of the medicine, the upper end of the graph bar shows the systolic blood pressure value, the lower end shows the diastolic blood pressure value, and the square mark indicates the pulse rate. The display example in FIG. 11 shows that the medication and blood pressure measurement were skipped on March 12.

Although the description will not be given, the contents on the display 314 as in FIG. 10 or 11 may be also printed out.

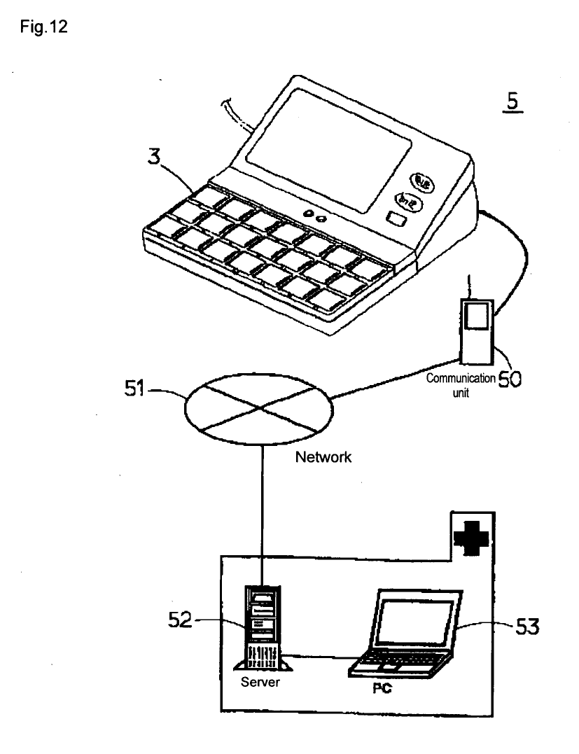

FIG. 12 shows a diagram showing an example of configuration of a medication management system 5 of the invention. Referring to FIG. 12, in the medication management system of the invention, a case of using the medication management case 3 with a blood pressure monitor will be described. The medication management system 5 of the invention comprises the medication management case 3 with a blood pressure monitor, a server 52 for receiving and accumulating information obtained in the medication management case 3 with a blood pressure monitor such as medication record and blood pressure measurement by way of a communication device 50 and network 51, and a personal computer 53 for browsing the information accumulated in the server 52, and entering updating of medication timing or other instructions.

The communication unit 50 is a device having means for transmitting and receiving data by way of the network 51, and it is realized by personal computer or cell phone. The network 51 connecting the communication unit 50 and server 52 is realized by the Internet, or network using dedicated line, wireless means, or other communication method. The server 52 and personal computer 53 in the medical institute may be connected by LAN (Local Area Network) or other dedicated line, or telephone line, wireless communication or other connection means. Although not shown in FIG. 12, it is more preferable to form a network in the medical institute by connecting to the personal computer at the pharmacy.

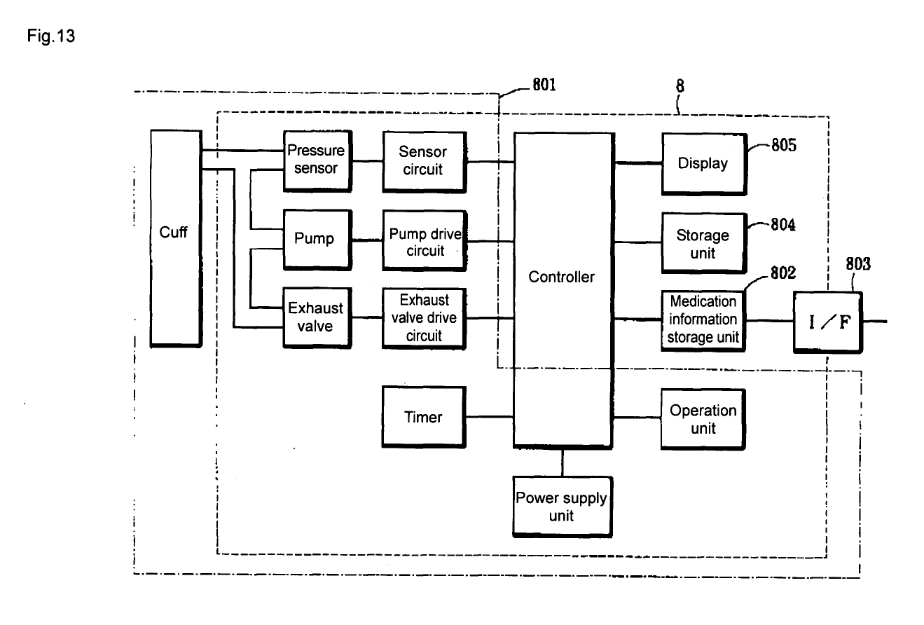

FIG. 13 is a functional block diagram showing a configuration of a blood pressure monitor 8 with a medication information display function capable of storing and displaying the medication information as an embodiment of a blood pressure monitor of the invention.

Referring to FIG. 13, it will be described below. A blood pressure measurement section 801 comprising a controller constituted of CPU and others, a cuff, a pressure sensor, a sensor circuit, a pump, a pump drive circuit, an exhaust valve, an exhaust valve drive circuit, a timer, a power supply unit and an operation unit is same as the configuration of a conventional blood pressure monitor; therefore, its description will not be given. This blood pressure monitor 8 with a medication information display function has a medication information storage unit 802, and this medication information storage unit 802 can receive and store the medication information, such as compliance with medication, type of the medicine taken, and date and time of medication, from outside by way of an I/F (interface) unit 803 capable of communicating with outside. The medication information can be received from outside by the Internet or other network using dedicated line, wireless means, or other communication method, or the medication information can be obtained by coupling with the medication management case 1 or 2 by a dedicated cable (not shown).

The storage unit 804 stores the blood pressures measured by the blood pressure measurement section 801 and the date and time of measurement, and the display unit 805 constituted of liquid crystal or the like displays not only the blood pressure values measured by the blood pressure measurement section 801 and the date and time of measurement, but also the medication information stored in the medication information storage means such as compliance with medication, type of the medicine taken, and date and time of medication. The information may be displayed on a screen same as in FIG. 10 or 11, or printed out on paper, or issued to outside although not shown.

As described herein, according to the invention, the medicine to be taken is indicated at every timing specified by the attending physician at a medical institute or the like, and the medicine can be taken without error or fail, and the medication information is all stored automatically, so that an accurate medication record is obtained.

Also according to the invention, the blood pressure measuring results such as systolic blood pressure value, diastolic blood pressure value, pulse rate, and date and time of measurement and the medication record such as the type of the medicine taken and the date and time of medication are simultaneously displayed in figure, graph or printer, and the blood pressure information and medication record indispensable for medicine therapy of hypertension can be understood simultaneously at a glance, and changes of blood pressure information and medication effects are easily determined, and clinically useful information can be obtained.

Also according to the invention, since the data can be transmitted and received with the information terminals installed at medical institute and others, and the attending physician at the medical institute can obtain the blood pressure information and reliable medication record as required, and acquire useful information for diagnosis, and further instruct medication schedule, so that an effective hypotensive medicine treatment can be executed.

Also according to the invention, not only the blood pressure information such as systolic blood pressure value, diastolic blood pressure value, pulse rate, and date and time of measurement, but also the medication information, such as compliance to medication, type of the medicine taken and the date and time of medication can be easily acquired, and the effects of medication on the blood pressures are easily to understand for the user of the blood pressure monitor, and useful information for daily blood pressure control and health management can be obtained.

What is claimed is:

1. A medicine case comprising:
medicine storage means for storing one or plural medicines to be taken each time by the user in divided portions;
medication instruction storage means for storing instruction of medication entered from outside;
medication instruction means for instructing the medicine to be taken from the divided portions stored in the medicine storage means on the basis of the medication instruction of the medication instruction storage means;
medication detection means for detecting taking of the medicine instructed by the medication instruction means;
storage output means for storing the medicine taking record detected by the medication detection means, and issuing to outside; and
display means for displaying the medicine taking record stored in the storage output means.

2. The medicine case according to claim 1, wherein
the medicine storage means is constituted of a plurality of compartments having lids for storing in divided portions according to each medicine taking timing by the user,
the medication instruction means instructs the compartment storing the medicine to be taken, and
the medication detection means detects opening and closing of the lid of the compartment.

3. The medicine case according to claim 1, wherein
the medicine storage means is constituted of a plurality of slits holding one pouch each of the medicine to be taken,
the medication instruction means instructs one or plural slits holding the pouches to be taken, and
the medication detection means detects taking of the pouch out of the slit.

4. A medicine case with a blood pressure measuring function comprising:
medicine storage means for storing one or plural medicines to be taken each time by the user in divided portions;
medication instruction storage means for storing instruction of medication entered from outside;
medication instruction means for instructing the medicine to be taken from the divided portions stored in the medicine storage means on the basis of the medication instruction of the medication instruction storage means;
medication detection means for detecting taking of the medicine instructed by the medication instruction means;
blood pressure measurement means for measuring the blood pressure of the user;
storage output means for storing the blood pressure value measured by the blood pressure measurement means and the medicine taking record detected by the medication detection means, and issuing to outside, and
display means for displaying the blood pressure value and the medicine taking record stored in the storage output means.

5. The medicine case with a blood pressure measuring function according to claim 4, wherein
the storage output means stores the systolic blood pressure value, diastolic blood pressure value, pulse rate and date and time of measurement measured by the blood pressure measurement means, and the medicine taking time detected by the medication detection means, and also issues to outside.

6. The medicine case with a blood pressure measuring function according to claim 4, wherein
the display means displays the systolic blood pressure value, diastolic blood pressure value, pulse rate, date and time of measurement, type of medicine, and medicine taking time stored in the storage output means, in list display, graph display, or printer display.

7. A medication management system comprising:
a medicine case with a blood pressure measuring function including
medicine storage means for storing one or plural medicines to be taken each time by the user in divided portions,
medication instruction storage means for storing instruction of medication entered from outside,
medication instruction means for instructing the medicine to be taken from the divided portions stored in the medicine storage means on the basis of the medication instruction of the medication instruction storage means,
medication detection means for detecting taking of the medicine instructed by the medication instruction means,
blood pressure measurement means for measuring the blood pressure of the user,
storage output means for storing the blood pressure value measured by the blood pressure measurement means and the medicine taking record detected by the medication detection means, and issuing to outside, and
display means for displaying the blood pressure value and the medicine taking record stored in the storage output means;
medication instruction input means for entering medication instruction into the medication instruction storage means; and
external display means for storing and displaying the storage information issued from the storage output means.

8. The medication management system according to claim 7, wherein
the medication instruction input means and external display means are terminal controllers installed at a medical institute, further comprising:
communication means for transmitting and receiving between the terminal controllers and the medicine case with a blood pressure measuring function via public lines.

9. A blood pressure monitor comprising:
blood pressure measurement means for measuring the blood pressure of the user; and
storage means for storing the blood pressure values and date and time of measurement measured by the blood pressure measurement means, the blood pressure monitor further comprising:
medication information storage means for receiving and storing medication information from outside; and
display means for displaying the medication information stored in the medication information storage means together with the blood pressure values and date and time of measurement stored in the storage means.
